(19) United States
(12) Patent Application Publication
(10) Pub. No.: US 2025/0238873 A1
(43) Pub. Date: Jul. 24, 2025

(54) SYSTEMS AND METHODS FOR AUTOMATED PRE-ADMINISTRATION OF HEALTHCARE PLAN PRE-APPROVED PHARMACY PRIOR AUTHORIZATIONS

(71) Applicant: Caper Berry, LLC, Chesterfield, MO (US)

(72) Inventors: Jay Baumohl, Chesterfield, MO (US); Erica Schulze, Richmond Heights, MO (US); Timothy J. King, Gig Harbor, WA (US); Charles E. Turco, Des Peres, MO (US)

(21) Appl. No.: 18/417,498

(22) Filed: Jan. 19, 2024

(51) Int. Cl. G06Q 40/08; G16H 40/20

(52) U.S. Cl. CPC G06Q 40/08; G16H 40/20

(57) ABSTRACT

Described herein are pharmacy claim transaction processing systems and methods. A method for pre-approval of a prior authorization includes receiving a pharmacy claim transaction message associated with a pharmacy claim transaction for a prescription that satisfies a prior authorization (PA) trigger condition, the message including a plan identifier and a prescriber identifier. The method includes querying a database with the prescriber identifier and plan identifier to access prescriber reference data, healthcare plan provisions, and jurisdictional regulations, as well as applying pre-approval rules to the prescriber reference data, healthcare plan provisions, and jurisdictional regulations, the pre-approval rules defining pre-approval conditions for pharmacy PAs. The method includes, when the pre-approval conditions are met, automatically appending an approval data element to the message to generate a PA approval message and transmitting the PA approval message. The method further includes writing a record to the database including whether the transaction satisfied the pre-approval conditions.

### FIELD

This disclosure relates generally to the field of electronic pharmacy claim processing and, more specifically, to the automated administration of healthcare plan pre-approved pharmacy prior authorization claims submitted over an electronic claims processing network.

### BACKGROUND

It is well known that some prescriptions for products or services provided at a pharmacy (e.g., medication, medical devices, medical supplies, laboratory orders) require a prior authorization (PA), based on provisions of a healthcare plan or insurer. Some common instances for requiring a PA may include, but are not limited to, medications prescribed for off-label or not yet approved uses, prescribed products having cheaper or more commonly available treatment alternatives, prescribed medical devices, prescribed medical supplies, prescription digital therapeutics, and/or medication-specific clinical protocols including step therapy. When a pharmacy PA is initiated, the prescriber must provide additional information to the healthcare plan or insurer, for the insurer to approve the prescription for coverage under the healthcare plan. This process commonly requires multiple phone calls or faxes and the transmission of unfilled and filled forms between parties and may require days or weeks of time and effort by multiple parties. In some cases, prescribers are not sure of what information is needed and may provide unnecessary information to the insurer. In many cases, the PA process results in delays in treatment, which can be significant and, at times, harmful to the patients.

Therefore, there is a need for an electronic prescription claims processing system and network that enables real-time automated administration of pharmacy prior authorization claims transactions qualifying for pre-approval or waiver.

### BRIEF DESCRIPTION

In one aspect, a computer-implemented method for automated administration of pre-approval of prescription product prior authorizations is provided. The method is implemented using a prior authorization pre-approval (paPA) computing device including a processor in communication with a memory, the paPA computing device in networked communication with a pharmacy switch server associated with a plurality of pharmacies and a plurality of pharmacy claims processors. The method includes: (a) receiving, from the pharmacy switch server, a pharmacy claim transaction message associated with a pharmacy claim transaction for a prescription that has satisfied a prior authorization (PA) trigger condition, the pharmacy claim transaction message including (i) a plan identifier associated with a healthcare plan under which the prescription is submitted at an originating pharmacy, and (ii) a prescriber identifier associated with a prescriber of the prescription; (b) querying a reference database with the prescriber identifier and the plan identifier to access prescriber reference data associated with the prescriber of the prescription, healthcare plan provisions of the healthcare plan, and jurisdictional regulations; (c) applying a set of pre-approval rules to the prescriber reference data, the healthcare plan provisions, and the jurisdictional regulations, the set of pre-approval rules defining pre-approval conditions for pharmacy PAs; (d) when the pre-approval conditions are met: (i) automatically appending an approval data element to the pharmacy claim transaction message to generate a PA approval message; and (ii) transmitting the PA approval message to the pharmacy switch server for forwarding to an originating pharmacy to fulfill the prescription or to a first pharmacy claims processor for further adjudication of the pharmacy claim transaction; and/or (e) writing a new record to the reference database including an indication of whether the pharmacy claim transaction for the prescription satisfied the pre-approval conditions.

In another aspect, a prior authorization pre-approval (paPA) computing device is provided. The paPA computing device includes at least one processor in communication with at least one memory device storing computer-executable instructions that, when executed by the at least one processor, cause the at least one processor to: (a) receive, from a pharmacy switch server, a pharmacy claim transaction message associated with a pharmacy claim transaction for a prescription that has satisfied a prior authorization (PA) trigger condition, the pharmacy claim transaction message including (i) a plan identifier associated with a healthcare plan under which the prescription is submitted at an originating pharmacy, and (ii) a prescriber identifier associated with a prescriber of the prescription, wherein the pharmacy switch server is associated with a plurality of pharmacies and a plurality of pharmacy claims processors; (b) query a reference database with the prescriber identifier and the plan identifier to access prescriber reference data associated with the prescriber of the prescription, healthcare plan provisions of the healthcare plan, and jurisdictional regulations; (c) apply a set of pre-approval rules to the prescriber reference data, the healthcare plan provisions, and the jurisdictional regulations, the set of pre-approval rules defining pre-approval conditions for pharmacy PAs; (d) when the pre-approval conditions are met: (i) automatically append an approval data element to the pharmacy claim transaction message to generate a PA approval message; and (ii) transmit the PA approval message to the pharmacy switch server for forwarding to an originating pharmacy to fulfill the prescription or to a first pharmacy claims processor for further adjudication of the pharmacy claim transaction; and/or (e) write a new record to the reference database including an indication of whether the pharmacy claim transaction for the prescription satisfied the pre-approval conditions.

### BRIEF DESCRIPTION OF THE DRAWINGS

FIGS. 1-5 show example embodiments of the methods and systems described herein.

FIG. 1 is a schematic diagram illustrating an example pharmacy claim processing system, including a prior authorization pre-approval (paPA) computing device, in accordance with the present disclosure.

FIG. 2 is a schematic diagram further illustrating the pharmacy claim processing system including the paPA computing device shown in FIG. 1.

FIG. 3 illustrates an example automated prior authorization processing method that may be implemented using the system shown in FIG. 1.

FIG. 4 illustrates an example server computing device that may be used with the system shown in FIG. 1.

FIG. 5 illustrates an example user computing device that may be used with the system shown in FIG. 1.

Like numbers in the Figures indicate the same or functionally similar components.

### DETAILED DESCRIPTION

The present disclosure relates to a pharmacy claim processing system that replaces existing prior authorization (PA) processing in certain situations, automating administration of healthcare plan pharmacy claims processing to eliminate inefficiencies while enhancing data consistency and usability. In particular, the pharmacy claim processing system includes a prior authorization pre-approval (paPA) computing device that facilitates the receipt and processing of pharmacy claims that satisfy PA trigger conditions, indicating a prior authorization for a prescription product (e.g., medication, medical devices, medical supply, laboratory order, etc.) is called for by the applicable healthcare plan. The paPA computing device applies data from the pharmacy claim and data from database of prescriber and plan reference data to a set of pre-approval rules. If the pre-approval rules are satisfied, then the PA is considered “pre-approved,” such that the requirement for the PA is waived, without requiring any further intervention from a prescriber, pharmacy, or other provider. In the context of the present application, “existing PA processing” may include manual and/or electronic PA processing, such as telephonic, fax, and paper-based processing routines that are conventionally handled in large part by persons. That is, the use of the term “manual,” when referring to existing PA processing, includes any electronic processing routines that are initiated, reviewed, or otherwise handled in part or in whole by a person.

In this way, the pharmacy claim processing system of the present disclosure eliminates the need for (existing) PA processing in many cases. This advancement facilitates a significant reduction in typical manual processes, which require not only time and effort by many persons (e.g., the prescriber, persons from the pharmacy, persons from the provider, persons from a pharmacy claims processor, etc.) but also significant computing resources, including the resources for back-and-forth messaging as well as the storage of unfilled and filled PA forms and, oftentimes, extraneous data requested from the prescriber. The pharmacy claim processing system including the paPA computing device also enables these PA preapprovals or waivers to be performed by leveraging the pharmacy claim processing ecosystem (e.g., including the pharmacy, a pharmacy switch server, and a pharmacy claims processor), limiting infrastructure changes required to enable PA preapprovals. The technical problems addressed by this system include at least one of: (i) significant time and effort required for existing and manual PA processing, which frequently results in delayed treatment; (ii) lack of data integration between prescribers and pharmacy claims processors; (iii) inability of prescribers to access or determine what information is needed to fulfill PA requirements; (iv) significant technology requirements for preparing, transmitting, and storing information between parties to fulfill a PA; and (v) delays in initiation of PA processing until other pharmacy claim adjudication is completed.

The methods and systems described herein may be implemented using computer programming or engineering techniques including computer software, firmware, hardware, or any combination or subset thereof, wherein the technical effects may be achieved by: (a) receiving, from a pharmacy switch server, a pharmacy claim transaction message associated with a pharmacy claim transaction for a prescription that has satisfied a prior authorization (PA) trigger condition, the pharmacy claim transaction message including (i) a plan identifier associated with a healthcare plan under which the prescription is submitted at an originating pharmacy, and (ii) a prescriber identifier associated with a prescriber of the prescription; (b) querying a reference database with the prescriber identifier and the plan identifier to access prescriber reference data associated with the prescriber of the prescription, healthcare plan provisions of the healthcare plan, and/or jurisdictional regulations; (c) applying a set of pre-approval rules to the prescriber reference data, the healthcare plan provisions, and the jurisdictional regulations, the set of pre-approval rules defining pre-approval conditions for pharmacy PAS; (d) when the pre-approval conditions are met: (i) automatically appending an approval data element to the pharmacy claim transaction message to generate a PA approval message; and (ii) transmitting the PA approval message to the pharmacy switch server for forwarding to an originating pharmacy to fulfill the prescription or to a first pharmacy claims processor for further adjudication of the pharmacy claim transaction; and/or (e) writing a new record to the reference database including an indication of whether the pharmacy claim transaction for the prescription satisfied the pre-approval conditions.

The resulting technical benefits achieved by this system include at least one of: (i) automated administration of healthcare plan pre-approved pharmacy prior authorizations, whether reimbursed through a pharmacy benefit or medical benefit of a healthcare plan; (ii) providing a software gateway between pharmacies, pharmacy switch servers, or pharmacy claims processors, that enables PAs to be pre-approved or waived according to jurisdictional regulations and/or healthcare plan provisions; (iii) real-time, automated PA processing; (iv) efficient and secure pharmacy claim processing; (v) enabling automated PA processing with a single pre-approval/waiver mechanism for all applicable prescriptions; and (vi) pre-approval/waiver of PAs before adjudication and/or during adjudication of a claim by a pharmacy claims processor. At least some of these technical benefits are achieved based on the unique location of the paPA computing device within the overall claim processing computing environment. Specifically, the association of the paPA computing device with the pharmacy switch server, as described herein, enables the pharmacy claim processing system to leverage existing connections and infrastructure between pharmacies, pharmacy claims processors, and pharmacy switch servers. The unique location of the paPA computing device, relative to the system, also enables the processing of incoming real-time claim transactions, to enable real-time and automated PA processing.

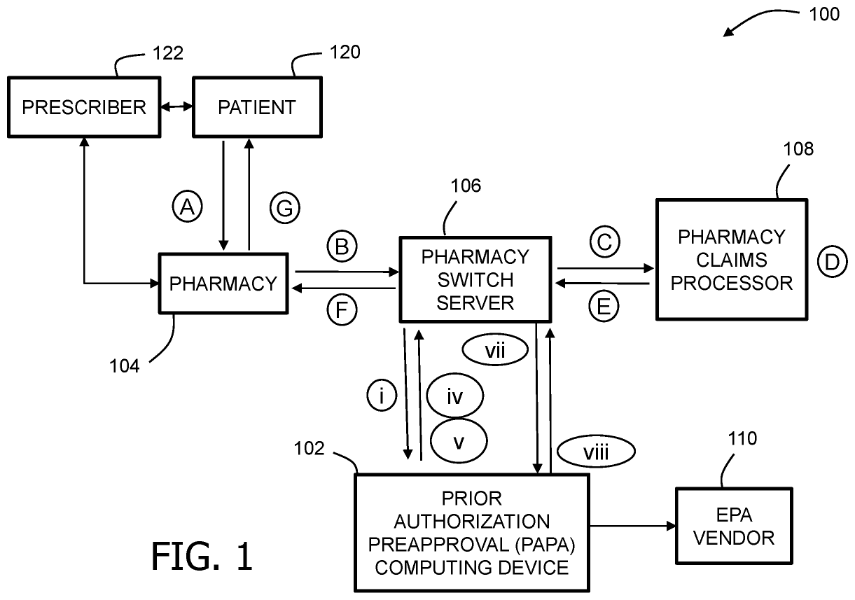

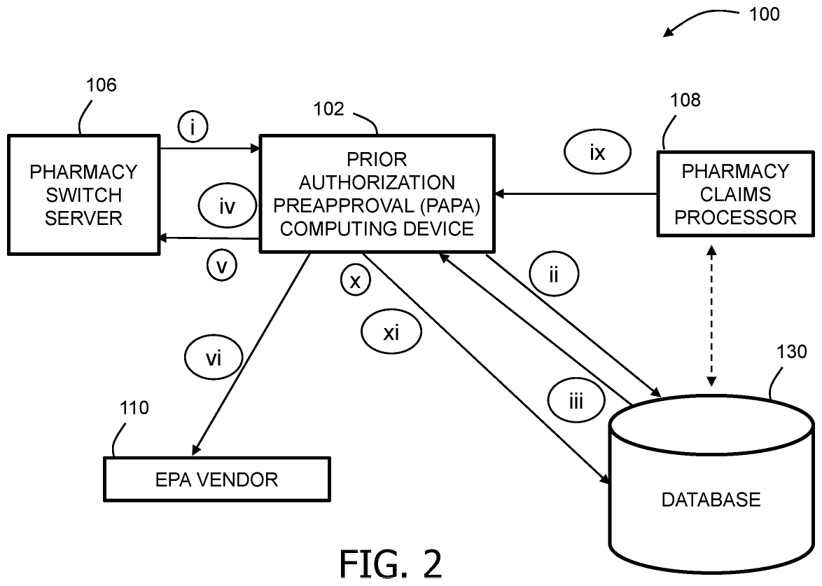

FIGS. 1 and 2 include a schematic diagram illustrating an example pharmacy claim processing system 100 for enabling PA pre-authorization in real-time during the claim transaction processing, under particular conditions, as well as an example pharmacy claim processing method implemented using the pharmacy claim processing system 100.

In the example embodiment, the pharmacy claim processing system 100 includes a prior authorization pre-approval (paPA) computing device 102, which facilitates at least a portion of the pharmacy claim processing methods described further herein. The paPA computing device 102 may include any suitable computing device(s), such as one or more server computing device(s), databases, cloud-based computing and/or storage systems, and/or any other device(s). Additionally, the paPA computing device 102 may include or implement tokenization and/or encryption schema during pharmacy claim processing and inter-device messaging, to ensure security of any personally identifiable information and/or other sensitive patient information. The paPA computing device 102 enables real-time PA processing, including PA pre-approval (also referred to herein as PA waiver) during claim transaction processing. “Real-time” refers to processes or routines that occur without substantial delay, such as within seconds or minutes. “Real-time PA pre-approval,” therefore, takes place within seconds or minutes of a pharmacy claim transaction being initiated. The paPA computing device 102 may be configured to receive, transmit, format, and re-format messages according to any suitable messaging standard for pharmacy claims processing, such as, but not limited to, NCPDP SCRIPT Standard.

The pharmacy claim processing system 100 further includes a plurality of pharmacies 104. It should be understood that although only one pharmacy 104 is shown, the functionality of the pharmacy claim processing system 100, particularly of the paPA computing device 102, is accessible to any number of pharmacies that are in networked communication with a pharmacy switch server 106, as described further herein. Additionally, the pharmacy claim processing system 100 includes a pharmacy claims processor server 108, which typically includes an administrator of prescription drug programs for commercial health plans, self-insured employer plans, Medicare Part D plans, the Federal Employees Health Benefits Program, and state government employee plans. The pharmacy claims processor 108 may include a pharmacy benefit manager. In at least some embodiments, the pharmacy claim processing system 100 also includes an electronic PA vendor computing device 110. The electronic PA vendor computing device 110 is configured to store and manage conventional PA forms that are sent to prescribers during existing PA processing and is connected to the prescriber 122 via an internet portal and/or an electronic health record (EHR). The electronic PA vendor computing device 110 is connected to the pharmacy claims processor 108 via an API call (e.g., via a utilization management device, not shown) or other established networked communication with data exchanged using the NCPDP SCRIPT standard.

Notably, pharmacies 104, pharmacy claims processors 108, and electronic PA vendor computing devices 110 are configured to function, in general, the same in the pharmacy claim processing system 100 as in conventional systems. That is, the pharmacy claim processing system 100 of the present disclosure provides the technical benefits and improvements described herein without a significant restructuring of existing entities, which enables the pharmacy claim processing system 100 substantially seamless functionality. It should be recognized, however, that the conventional functionality of the electronic PA vendor computing device 110 may be employed less frequently, as the automatic pre-approval of prior authorization may render such functionality moot in many cases.

The pharmacy claim processing system 100 also includes a networked pharmacy switch server 106, which is configured to process claim transaction messages and function as a messaging “gateway” between various entities within the pharmacy claim processing system 100, including the pharmacy 104, the pharmacy claims processor 108, and, in the example embodiment, the paPA computing device 102. The pharmacy switch server 106 may include, in some cases, a medical claims clearinghouse. It is generally understood that certain pharmacies 104 and/or pharmacy claims processors 108 may be associated with one particular pharmacy switch server 106, for instance, based on existing contractual relationships, messaging infrastructure, and the like. In the example embodiment, the paPA computing device 102 may be associated with one pharmacy switch server 106 or with multiple pharmacy switch servers 106, where each pharmacy switch server 106 serves a different set of pharmacies 104 and pharmacy claims processors 108.

At least some communication channels and/or data exchange described herein, such as between various components of the pharmacy claim processing system 100, may be implemented using an API ecosystem to enable direct data integration between those components. Additionally, certain forms available to facilitate this data connection may be implemented using standardized form types (e.g., a JAVASCRIPT library) to enable direct integration.

In operation of the pharmacy claim processing system 100, a patient 120 is prescribed a particular product (e.g., medical, medical device, etc.) by prescriber 122. The prescription is provided (A) to the pharmacy 104 to be filled. In some instances, the prescriber 122 transmits the prescription to the pharmacy 104 electronically; in other instances, the patient 120 is provided with a paper prescription that the patient 120 then physically brings to the pharmacy 104 for fulfillment. At the pharmacy 104, the prescription is processed by, in part, submitting a pharmacy claim transaction. In particular, the pharmacy 104 transmits (B) an electronic claim transaction message to the pharmacy switch server 106. This transmission (B) may be performed using an electronic software platform. The claim transaction message includes a plurality of data elements including, but not limited to, a plan identifier of the healthcare plan of the patient 120, a prescriber identifier of the prescriber 122, and a transaction identifier that functions to relate all messages associated with the same pharmacy claim transaction. The claim transaction message also includes a prescription identifier of the product prescribed, and may include additional data elements such as a patient identifier, time/date stamp associated with the origination of the claim transaction at the pharmacy 104, a pharmacy claims processor identifier of the particular pharmacy claims processor 108 responsible for managing the healthcare plan, a pharmacy identifier of the pharmacy 104, prescription history (e.g., fill date(s), fill status(es)), and a PA status indicator, in some instances.

As described above, the pharmacy switch server 106 functions as a gateway from the pharmacy 104 to other entities and data sources. The pharmacy switch server 106 transmits the claim transaction message, or data elements parsed therefrom, to the various entities to perform claim processing.

For instance, in typical claim transaction processing, the pharmacy switch server 106 transmits (C) the claim transaction message to the pharmacy claims processor server 108 for processing (D) of the pharmacy claim transaction (e.g., via an API call or other established networked communication). This processing (D) can include, for example, a verification or authentication of the validity of the eligibility of the patent 120 and, therefore, of the prescription, as well as insurance-related processing to determine the patient's financial obligations (e.g., a copay, coinsurance). The pharmacy claims processor 108 transmits (E) an electronic claim response message, also referred to as an adjudication message, back to the pharmacy switch server 106. The adjudication message includes data elements corresponding to the results of the processing performed by the pharmacy claims processor 108. The pharmacy switch server 106 then forwards (F) the adjudication message back to the pharmacy 104, such that the pharmacy 104 can process (G) the prescription, take payment from the patient 120, and the like. Alternatively, where the processing indicates that a PA is required and has not already been provided, conventionally, the pharmacy claims processor 108 transmits (E) the adjudication message back to the pharmacy 104, where the patient 120 is informed (G) that their prescription cannot be fulfilled until the PA requirements are met by the prescriber 122.

In accordance with the present disclosure, the pharmacy switch server 106 is further configured to identify, from the claim transaction message, that the pharmacy claim transaction has a prior authorization requirement associated therewith, before the claim transaction message is forwarded (C) to the pharmacy claims processor 108 for adjudication (D). In one embodiment, the pharmacy switch server 106 parses a prescription identifier and a plan identifier from the claim transaction message. Certain combinations of prescription identifiers and plan identifiers trigger a PA condition, indicating that a particular plan and/or prescriber requires prior authorization to fill prescriptions of that product and be eligible for reimbursement. The pharmacy switch server 106 queries a list or database that stores these combinations and, when a “hit” or match is returned, the pharmacy switch server 106 detects that particular pharmacy claim transaction has a prior authorization requirement. Upon such detection, the pharmacy switch server 106 transmits (i) the claim transaction message to the paPA computing device 102 for automated PA processing.

Turning to FIG. 2, functionality of the paPA computing device 102 within the pharmacy claim processing system 100 is shown, starting with the paPA computing device 102 receiving (i) the claim transaction message from the pharmacy switch server 106. As described above, the pharmacy claim transaction message includes a plan identifier associated with a healthcare plan under which the prescription is submitted at the pharmacy 104, and a prescriber identifier associated with the prescriber 122.

The paPA computing device 102 queries (ii) a reference database 130 with the prescriber identifier and the plan identifier. The reference database 130, described further herein, stores prescriber reference data associated with a plurality of prescribers-including the prescriber 122—as well as healthcare plan provisions of a plurality of healthcare plans—including the healthcare plan of the patient 120. The reference database 130 returns (iii) the relevant prescriber reference data and healthcare provisions.

In the example embodiment, the paPA computing device 102 and/or the reference database 130 also store a plurality of pre-approval rules, which define pre-approval conditions for the various PAs that may be required by the healthcare plan provisions. The pre-approval conditions may vary based on the healthcare plan provisions as well as the jurisdiction in which the patient 120 is residing or the prescriber 122, the pharmacy 104, or the provider is operating. In some example embodiments, the pre-approval conditions include a minimum threshold number and/or percentage of PAs that have been approved, per a prescriber, over a predefined reference period of time. The predefined reference period of time may be static or rolling, and may be six months, nine months, one year, or any other applicable period of time. The minimum threshold percentage of PAs may be 100%, or may be another threshold value such as 99%, 95%, 90%, or any other value, which is based on jurisdictional regulations but may also vary based on healthcare plan provisions. The minimum threshold number of PAs—that is, the volume of prescriptions requiring PAs prescribed by a particular prescriber—may vary based on jurisdictional regulations and/or may vary based on healthcare plan provisions. Additional or alternative pre-approval conditions may exist having pre-approval rules associated therewith. For example, the above-described conditions may need to be met on a per-prescriber, per-provider, and/or per-healthcare plan basis, such that the prescriber's prescriptions with PAs that were approved under one healthcare plan may or may not apply towards the minimum threshold values for another healthcare plan.

The paPA computing device 102 applies the retrieved data (i.e., the prescriber reference data and the healthcare plan provisions) to the pre-approval rules, to determine whether the pharmacy claim transaction for the prescription meets the pre-approval conditions. For instance, with the above example of pre-approval conditions, the paPA computing device 102 determines whether the prescriber 122 has met the minimum threshold number and/or percentage of previously approved PAs over the reference period of time.

The paPA computing device 102 is configured to append a data element to the claim transaction message, where the data element is based on and indicates whether the pre-approval conditions are met. When the pre-approval conditions are met, the paPA computing device 102 automatically appends an approval data element to the claim transaction message, thereby generating a PA approval message. The PA approval message indicates that the PA has been pre-approved, or, stated differently, the need for the PA has been waived. The paPA computing device 102 transmits (iv) the PA approval message back to the pharmacy switch server 106. The pharmacy switch server 106 then proceeds with further processing of the pharmacy claim transaction as normal, without any further PA processing (in particular, without any additional manual intervention and/or messaging) as shown in FIG. 1. In one example embodiment, in which the claim transaction message is intercepted by the pharmacy switch server 106 and transmitted to the paPA computing device 102, the pharmacy switch server 106 proceeds to transmit (C) the claim transaction message to the pharmacy claims processor 108. More specifically, the pharmacy switch server 106 transmits (C) the PA approval message to the pharmacy claims processor 108 or transmits the claim transaction message with a flag or additional data element that communicates to the pharmacy claims processor 108 that PA pre-approval or waiver has been completed.

When the pre-approval conditions are not met, the paPA computing device 102 may take no action, passing the claim to the pharmacy switch server 106 and the pharmacy claims processor 108. In some instances, the pharmacy switch server 106 then proceeds to initiate the existing or conventional (e.g., manual) PA processing for the prescription. In some instances, the pharmacy switch server 106 transmits the PA rejection message to the pharmacy claims processor 108, and the pharmacy claims processor 108 adjudicates the claim transaction as normal (that is, initiating manual PA processing and review). In some embodiments, the paPA computing device 102 initiates conventional PA processing and review by transmitting (vi) an initiation message to the electronic PA vendor computing device 110 and/or to pharmacy claims processor 108.

In some embodiments of the present disclosure, the pharmacy switch server 106 is further configured to identify, from the claim transaction message, that the pharmacy claim transaction has a prior authorization requirement associated therewith, after the claim transaction has been adjudicated (D) by the pharmacy claims processor 108. In some instances, the pharmacy claims processor 108 determines a PA is required for the prescription, and transmits (E) the adjudication message back to the pharmacy switch server 106. The pharmacy switch server 106 parses the adjudication message and detects that particular pharmacy claim transaction has a prior authorization requirement. Upon such detection, the pharmacy switch server 106 transmits (vii) the adjudication to the paPA computing device 102.

In some instances, the paPA computing device 102 records the results of the adjudication by the pharmacy claims processor 108 in the reference database 130. These records may influence the pre-approved status of prescribers, for example. In some instances, the paPA computing device 102 may query (ii) the reference database 130 and determine a PA has been approved or waived for this particular claim transaction, based on the records in the reference database 130, and may append an appeal data element to the adjudication message before forwarding (viii) the (modified) adjudication message back to the pharmacy switch server 106. The pharmacy switch server 106 may then communicate the appeal data element back to the pharmacy claims processor 108 or to the pharmacy 104 for further review. In other instances, the paPA computing device 102 may query (ii) the reference database 130 and confirm the conclusion of the pharmacy claims processor 108 that additional PA processing and review are needed. The paPA computing device 102 may append a secondary rejection data element to the adjudication message before forwarding (viii) the (modified) adjudication message back to the pharmacy switch server 106. The paPA computing device 102 may additionally initiate (vi) the conventional PA processing with the electronic PA vendor computing device 110 and notify the pharmacy switch server 106 and/or the pharmacy claims processor 108 of the same.

In addition, FIG. 2 depicts internal functionality of the paPA computing device 102 to build and update the reference database 130. In particular, the paPA computing device 102 is configured to receive (ix) reference data from a plurality of pharmacy claims processors 108 and/or a plurality of pharmacy switch servers 106. The paPA computing device 102 stores (x) the reference data in the reference database 130. The reference data includes prescriber reference data, which is unique to each prescriber, as well as healthcare plan provisions, which are unique to each provider, and jurisdictional regulations, which may be defined relative to jurisdiction (e.g., country, state, county, city, etc.). The reference data may include, but is not limited to, providerNational Provider Identifiers (NPIs), pharmacy NPIs, product National Drug Codes (NDC), healthcare plan identifiers, member identifiers (including masked or encrypted member identifiers), prescription or claim histories (including prescription numbers from adjudicated/completed pharmacy claims, PA history and status, fill/refill data, transaction/claim status, quantity, day(s) supply, diagnosis code), prescriber location, pharmacy location, patient location, and drug formulary status (which may include a binary indicator that a prescribed product is/is not subject to PA). The reference data may be indexed by prescriber (or practice), provider, and/or jurisdiction, such that reference data that is commonly relevant to intersection(s) of parties can be retrieved by a prescriber, provider, and/or jurisdictional index.

In some embodiments, one or more pharmacy claims processors 108 may directly access the reference database 130 to store data, retrieve data, and/or update records. For example, a pharmacy claims processor 108 may perform “batch” uploads of transaction data for adjudicated transactions on a periodic basis, such as every night, every week, etc.

The paPA computing device 102 is configured to generate pre-approval rules based on the pre-approval conditions set by healthcare plan provisions and/or jurisdictional regulations. In some instances, the pre-approval conditions are defined and stored by the pharmacy claims processor 108. The paPA computing device 102 retrieves any pre-approval conditions and generates corresponding pre-approval rules, which may be formatted or embodied as algorithms or routines that take input data and output a result. In other embodiments, the pharmacy claims processor 108 stores pre-approval conditions and corresponding pre-approval rules. The paPA computing device 102 retrieves pre-approval rules from the reference database 130 during claim transaction processing, to apply retrieved reference data that is applicable to a subject transaction to the pre-approval rules. It should be understood that the pre-approval rules may be stored locally at the paPA computing device 102 without departing from the scope of the present disclosure.

In addition, the paPA computing device 102 is configured to determine and store a status for each prescriber that is represented in the reference database. This status may be referred to as a “gold card” status, an “approved” status, or “pre-approved PA” status, which indicates that the corresponding prescriber has already met the pre-approval conditions for a particular prescribed product with a particular jurisdiction and/or healthcare plan. When this status is detected, additional processing by the paPA computing device 102 may be avoided. For example, when the paPA computing device 102 queries (ii) the reference database 130 during PA processing of a pharmacy claim transaction, if the reference database 130 returns an approved status for the prescriber/prescribed product combination of the claim transaction, the paPA computing device 102 may not access the pre-approval rules. Instead, the paPA computing device 130 appends the approval data element (and/or the approved status or indicator thereof) to the claim transaction message before sending it back (iv) to the pharmacy switch server 106. In such cases, the PA processing performed by the paPA computing device 102 is even further streamlined.

The paPA computing device 102 may additionally determine and store a status progress indicator for each prescriber represented in the reference database 130. The status progress indicator may indicate a prescriber's progress toward achieving the approved status, based on, for example, a number of approved PAs or a percentage of approved PAs associated with the prescriber.

When a transaction is completed, the paPA computing device 102 is configured to update (xi) the reference database 130 based on the outcome of the claim transaction (e.g., whether the PA was pre-approved/waived or not). The paPA computing device 102 is configured to write and store a record for each claim transaction. Additionally, the paPA computing device 102 may be configured to update reference data or other records, after each transaction or periodically (e.g., daily, weekly, etc.). For example, the paPA computing device 102 may update the status progress indicator and/or status, as applicable, for a prescriber after a transaction is completed.

The reference database 130 therefore maintains a robust data set that can be leveraged (e.g., by the paPA computing device 102 or a pharmacy claims processor 108) to make conclusions and analyze trends beyond what was previously enabled. For example, the records stored in reference database 130 can be used to monitor claim transaction rejections (e.g., “reject 75s” or “National Council for Prescription Drug Programs (NCPDP) code 75 claims,” representing a rejection of a claim because a PA is required), to understand assignment of these rejections based upon cost, clinical efficacy, safety profile, and/or other medical requirements (e.g., step therapy), as well as the rejection rates on a plan, prescriber, or jurisdictional basis. The records may be leveraged (e.g., by a prescriber or practice, or by a pharmacy claims processor 108 or provider) to track and monitor progression towards a PA approved status for a particular prescriber or practice relative to one or more prescribed products. The records may be leveraged (e.g., by a pharmacy claims processor 108 or provider, or another third party) to generate and monitor fraud, waste, and abuse (FWA) metrics, for example, based on the absolute level of prescribing activity across multiple activity dimensions. The reference database 130, and the data stored therein, may be accessible to parties within the pharmacy claim processing system 100, for example, via a web-accessible portal enabled by an API connection, or by some other software platform. The data may be made available to certain parties, including entities outside of the pharmacy claim processing system 100, in an aggregated or other anonymized format, such that no personally identifiable or otherwise sensitive information is vulnerable.

The pharmacy claim processing system 100, including the paPA computing device 102, of the present disclosure therefore is configured to automate PA pre-approval/waiver status for prescription products in accordance with healthcare plan provisions as well as jurisdictional regulations. The system and methods described herein are not limited to the particular example embodiment of an initial pharmacy claim, and are applicable to many other situations including: (a) requests submitted via a prior authorization request form, whether in a manual or automated fashion; (b) requests submitted prospectively via an electronic health record (EHR), healthcare plan PA portal, pharmacy claims processor PA portal, or ePA vendor portal, and (c) retrospective requests submitted by a pharmacy after an initial claim submission is rejected.

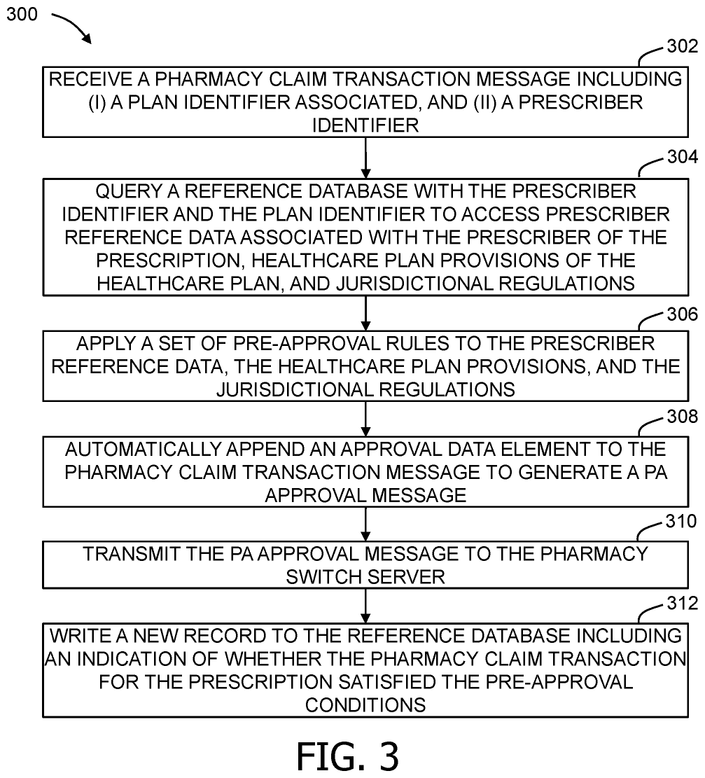

FIG. 3 is an example flow diagram illustrating a method 300 for pre-approval of pharmacy product prior authorizations. The method 300 may be implemented using one or more components of the pharmacy claim processing system 100 (shown in FIG. 1), including the paPA computing device 102 (also shown in FIG. 1).

The method 300 includes, in the example embodiment, receiving 302, from a pharmacy switch server (e.g., pharmacy switch server 106, shown in FIG. 1), a pharmacy claim transaction message associated with a pharmacy claim transaction for a prescription that has satisfied a prior authorization (PA) trigger condition. The pharmacy claim transaction message includes (i) a plan identifier associated with a healthcare plan under which the prescription is submitted at an originating pharmacy (e.g., the pharmacy 104, shown in FIG. 1), and (ii) a prescriber identifier associated with a prescriber of the prescription.

The method 300 also includes querying 304 a reference database (e.g., the reference database 130, shown in FIG. 2) with the prescriber identifier and the plan identifier to access prescriber reference data associated with the prescriber of the prescription and healthcare plan provisions of the healthcare plan. Querying 304 may also include querying the reference database for jurisdictional regulations.

The method 300 further includes applying 306 a set of pre-approval rules to the prescriber reference data, the healthcare plan provisions, and the jurisdictional regulations, the set of pre-approval rules defining pre-approval conditions for pharmacy PAs. The method 300 includes, when the pre-approval conditions are met, automatically appending 308 an approval data element to the pharmacy claim transaction message to generate a PA approval message and transmitting 310 the PA approval message to the pharmacy switch server to a first pharmacy claims processor for further adjudication of the pharmacy claim transaction. In some alternative embodiments, the transmitting 310 may include transmitting the PA approval message to the pharmacy switch server for forwarding to an originating pharmacy to fulfill the prescription.

The method 300 further includes writing 312 a new record to the reference database including an indication of whether the pharmacy claim transaction for the prescription satisfied the pre-approval conditions.

The method 300 may include additional, fewer, or alternative steps, in other embodiments. For example, in some embodiments, the method 300 also includes building (e.g., by the paPA computing device) the reference database based on prescriber reference data provided by each of the plurality of pharmacy claims processors networked to the pharmacy switch server and/or a plurality of pharmacy switch servers including the pharmacy switch server.

In some embodiments, the method 300 includes generating (e.g., by the paPA computing device) the set of pre-approval rules based on jurisdictional regulations and healthcare plan provisions provided by each of the plurality of pharmacy claims processors and/or a plurality of pharmacy switch servers including the pharmacy switch server.

In some embodiments, the method 300 includes, when the pre-approval conditions are not met, automatically appending (e.g., by the paPA computing device) a PA initiation data element to the pharmacy claim transaction message to generate a PA initiation message, and transmitting the PA rejection message to the pharmacy switch server for forwarding to the originating pharmacy, to the first pharmacy claims processor to initiate existing, manual PA processing of the pharmacy claim transaction, or to an electronic PA vendor to initiate manual PA processing of the pharmacy claim transaction by transmitting one or more PA forms to the prescriber.

In some embodiments, the method 300 includes assigning (e.g., by the paPA computing device) a transaction identifier associated with the pharmacy claim transaction, wherein the new record includes the transaction identifier.

In some embodiments, the method 300 includes storing, in the reference database (e.g., by the paPA computing device), for each respective prescriber of a plurality of prescribers including the prescriber, at least one of: a number of PAs approved in a reference period of time before the pharmacy claim transaction was submitted, or a percentage of PAs approved in the reference period of time. This data may be further indexed according to each respective prescribed product of a plurality of prescribed products, each respective jurisdiction of a plurality of jurisdictions, and/or respective provisions of each healthcare plan of a plurality of healthcare plans. In some such embodiments, the method 300 also includes storing, in the reference database, a pre-approval rule of the set of pre-approval rules defining a minimum threshold number of PAs approved or a minimum percentage of PAs approved (e.g., as established using at least one of the healthcare plan provisions or jurisdictional regulations). In some cases, the method 300 includes storing, in the reference database, the respective healthcare plan provisions for each of a plurality of healthcare plans, a national provider identifier, a plurality of prescription identifiers or prescribed product identifiers, a quantity, a quantity per days value, and a number of refills. The method 300 may also include generating one or more pre-approval rules of the set of pre-approval rules based on the healthcare plan provisions.

In some embodiments, the method 300 includes updating (e.g., by the paPA computing device) the prescriber reference data with the new record or, in some embodiments, when the pre-approval conditions are met, assigning a prescriber approved status to the prescriber reference data. In some cases, the method 300 also includes receiving, from the pharmacy switch server, a second pharmacy claim transaction message associated with a second pharmacy claim transaction for a prescription for a same prescribed product as the first pharmacy claim transaction and prescribed by the same prescriber as the first pharmacy claim transaction. The method 300 may further include querying the reference database with the prescriber identifier to access the prescriber reference data associated with the prescriber, detecting the prescribed approved status in the prescriber reference data, and automatically appending an approval data element to the second pharmacy claim transaction message to generate a second PA approval message.

In some embodiments, the method 300 includes monitoring, for the prescriber (and/or any other number of prescribers), a progress toward at least one prescriber approved status using the prescriber reference data (e.g., for each prescribed product/plan/jurisdiction, as described herein).

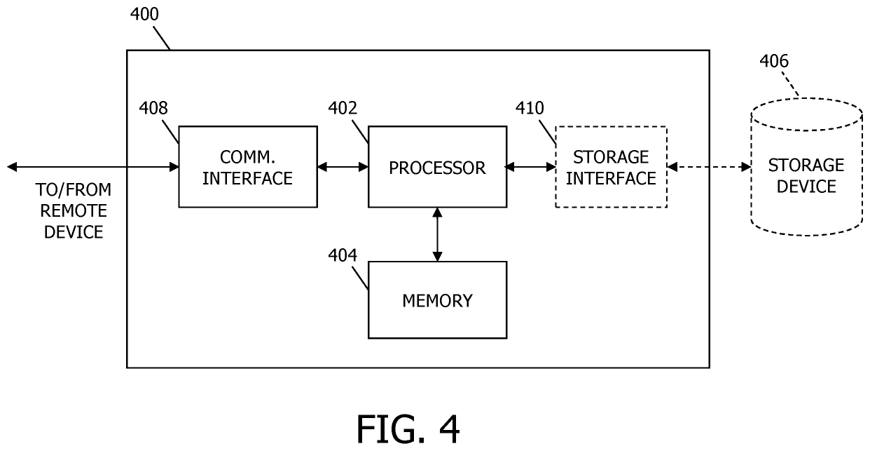

FIG. 4 shows an example configuration of a server system 400. The server system 400 may include, but is not limited to, the paPA computing device 102, the pharmacy switch 106, the pharmacy claims processor server 108, and the electronic PA vendor computing device 110 (all shown in FIG. 1), and/or computing device(s) associated with any party to the pharmacy claim processing system 100 (also shown in FIG. 1).

The server system 400 includes a processor 402 for executing instructions. Instructions may be stored in a memory area 404, for example. The processor 402 may include one or more processing units (e.g., in a multi-core configuration) for executing instructions. The instructions may be executed within a variety of different operating systems on the server system 400, such as UNIX, LINUX, Microsoft Windows®, etc. More specifically, the instructions may cause various data manipulations on data stored in the memory 404 and/or in a storage device 406 (e.g., create, read, update, and delete procedures). It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or other suitable programming languages, etc.).

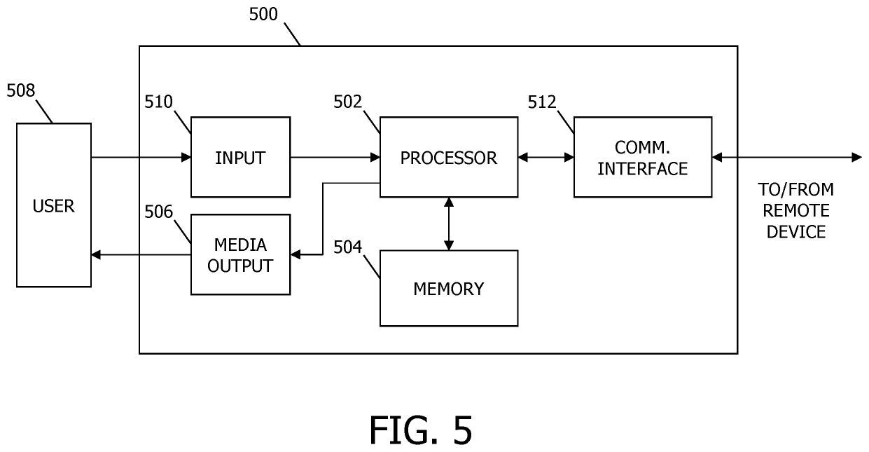

The processor 402 is operatively coupled to a communication interface 408 such that the server system 400 is capable of communicating with a remote device such as a user system 500 (shown in FIG. 5) or another server system 400. The processor 402 may also be operatively coupled to the storage device 406 (e.g., the database 130). The storage device 406 is any computer-operated hardware suitable for storing and/or retrieving data. In some embodiments, the storage device 406 is integrated in the server system 400. In other embodiments, the storage device 406 is external to the server system 400. For example, the server system 400 may include one or more hard disk drives as the storage device 406. In other embodiments, the storage device 406 is external to the server system 400 and may be accessed by a plurality of server systems 400. For example, the storage device 406 may include multiple storage units such as hard disks or solid-state disks in a redundant array of inexpensive disks (RAID) configuration. The storage device 406 may include a storage area network (SAN) and/or a network attached storage (NAS) system.

In some embodiments, the processor 402 is operatively coupled to the storage device 406 via a storage interface 410. The storage interface 410 is any component capable of providing the processor 402 with access to the storage device 406. The storage interface 410 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing the processor 402 with access to the storage device 406.

The memory area 404 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

FIG. 5 illustrates an example configuration of a user system 500, such as the pharmacy 104, the pharmacy claims processor 108, or the electronic PA vendor computing device 110. In the example embodiment, the user system 500 includes a processor 502 for executing instructions. In some embodiments, executable instructions are stored in a memory area 504. The processor 502 may include one or more processing units, for example, a multi-core configuration. The memory area 504 is any device allowing information such as executable instructions and/or written works to be stored and retrieved. The memory area 504 may include one or more computer readable media.

The user system 500 also includes at least one media output component 506 for presenting information to a user 508 (e.g., the patient 120, the prescriber 122, a person at the pharmacy 104, etc.). The media output component 506 is any component capable of conveying information to the user 508. In some embodiments, the media output component 506 includes an output adapter such as a video adapter and/or an audio adapter. An output adapter is operatively coupled to the processor 502 and operatively connectable to an output device such as a display device, a liquid crystal display (LCD), organic light emitting diode (OLED) display, or “electronic ink” display, or an audio output device, a speaker or headphones.

In some embodiments, the user system 500 includes an input device 510 for receiving input from the user 508. The input device 510 may include, for example, a keyboard, a pointing device, a mouse, a stylus, a touch sensitive panel, a touch pad, a touch screen, a gyroscope, an accelerometer, a position detector, or an audio input device. A single component such as a touch screen may function as both an output device of the media output component 506 and the input device 510. The user system 500 may also include a communication interface 512, which is communicatively connectable to a remote device (e.g., the paPA computing device 102, the pharmacy claims processor 108, both shown in FIG. 1). The communication interface 512 may include, for example, a wired or wireless network adapter or a wireless data transceiver for use with a mobile phone network, Global System for Mobile communications (GSM), 3G, or other mobile data network or Worldwide Interoperability for Microwave Access (WIMAX).

Stored in the memory area 504 are, for example, computer readable instructions for providing a user interface to the user 508 via the media output component 506 and receiving and processing user input from the input device 510. A user interface may include, among other possibilities, a web browser and client application (“app”). Web browsers enable users, such as the user 508, to display and interact with media and other information typically embedded on a web page or a website.

As will be appreciated based on the foregoing specification, the above-described embodiments of the disclosure may be implemented using computer programming or engineering techniques including computer software, firmware, hardware or any combination or subset thereof, wherein the technical effect is to enable the automated waiver or pre-approval of prior authorizations before, during, or after adjudication of a pharmacy claim by a pharmacy claims processor. Any such resulting program, having computer-readable code means, may be embodied or provided within one or more computer-readable media, thereby making a computer program product, (i.e., an article of manufacture), according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

In an example embodiment, the system is executed on a single computer system, without requiring a connection to a server computer. In a further embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independently and separate from other components and processes described herein. Each component and process can also be used in combination with other assembly packages and processes.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are example only and are thus not limiting as to the types of memory usable for storage of a computer program.

As used herein, an element or step recited in the singular and proceeded with the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

Disjunctive language such as the phrase “at least one of X, Y, or Z,” unless specifically stated otherwise, is generally intended, within the context presented, to disclose that an item, term, etc. may be either X, Y, or Z, or any combination thereof (e.g., X, Y, and/or Z). Likewise, conjunctive language such as the phrase “at least one of X, Y, and Z,” unless specifically stated otherwise, is generally intended, within the context presented, to disclose at least one of X, at least one of Y, and at least one of Z.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer-implemented method for automated administration of pre-approval of pharmacy product prior authorizations, the method implemented using a prior authorization pre-approval (paPA) computing device comprising a processor in communication with a memory, the paPA computing device in networked communication with a pharmacy switch server associated with a plurality of pharmacies and a plurality of pharmacy claims processors, the method comprising:
receiving, from the pharmacy switch server, a pharmacy claim transaction message associated with a pharmacy claim transaction for a prescription that has satisfied a prior authorization (PA) trigger condition, the pharmacy claim transaction message including (i) a plan identifier associated with a healthcare plan under which the prescription is submitted at an originating pharmacy, and (ii) a prescriber identifier associated with a prescriber of the prescription;
querying a reference database with the prescriber identifier and the plan identifier to access prescriber reference data associated with the prescriber of the prescription, healthcare plan provisions of the healthcare plan, and jurisdictional regulations;
applying a set of pre-approval rules to the prescriber reference data, the healthcare plan provisions, and the jurisdictional regulations, the set of pre-approval rules defining pre-approval conditions for pharmacy PAS;
when the pre-approval conditions are met:
automatically appending an approval data element to the pharmacy claim transaction message to generate a PA approval message; and
transmitting the PA approval message to the pharmacy switch server for forwarding to an originating pharmacy to fulfill the prescription or to a first pharmacy claims processor for further adjudication of the pharmacy claim transaction; and
writing a new record to the reference database including an indication of whether the pharmacy claim transaction for the prescription satisfied the pre-approval conditions.

2. The computer-implemented method of claim 1, further comprising building the reference database based on prescriber reference data provided by at least one of: (i) each of the plurality of pharmacy claims processors, or (ii) a plurality of pharmacy switch servers including the pharmacy switch server.

3. The computer-implemented method of claim 1, further comprising generating the set of pre-approval rules based on jurisdictional regulations and healthcare plan provisions provided by at least one of: (i) each of the plurality of pharmacy claims processors, or (ii) a plurality of pharmacy switch servers including the pharmacy switch server.

4. The computer-implemented method of claim 1, further comprising:
when the pre-approval conditions are not met:
automatically appending a PA initiation data element to the pharmacy claim transaction message to generate a PA initiation message; and
transmitting a PA rejection message to the pharmacy switch server for forwarding to the originating pharmacy, to the first pharmacy claims processor to initiate manual PA processing of the pharmacy claim transaction, or to an electronic PA vendor to initiate manual PA processing of the pharmacy claim transaction by transmitting one or more PA forms to the prescriber.

5. The computer-implemented method of claim 1, further comprising assigning a transaction identifier associated with the pharmacy claim transaction, wherein the new record includes the transaction identifier.

6. The computer-implemented method of claim 1, further comprising storing, in the reference database, for each respective prescriber of a plurality of prescribers including the prescriber, at least one of: a number of PAs approved in a reference period of time before the pharmacy claim transaction was submitted, or a percentage of PAs approved in the reference period of time, for each respective prescribed product of a plurality of prescribed products, for each respective jurisdiction of a plurality of jurisdictions, and for respective provisions of each healthcare plan of a plurality of healthcare plans.

7. The computer-implemented method of claim 6, further comprising storing, in the reference database, a pre-approval rule of the set of pre-approval rules defining a minimum threshold number of PAs approved or a minimum percentage of PAs approved as established using at least one of the healthcare plan provisions or jurisdictional regulations.

8. The computer-implemented method of claim 6, further comprising:
storing, in the reference database, the respective healthcare plan provisions for each of a plurality of healthcare plans, the respective jurisdictional provisions for each of a plurality of jurisdictions, a national provider identifier, a plurality of prescription drug identifiers, a quantity, a quantity per days value, and a number of refills; and
generating one or more pre-approval rules of the set of pre-approval rules based on the healthcare plan provisions.

9. The computer-implemented method of claim 1, further comprising updating the prescriber reference data with the new record.

10. The computer-implemented method of claim 1, wherein the pharmacy claim transaction is a first pharmacy claim transaction, the method further comprising:
when the pre-approval conditions are met, assigning a prescriber approved status to the prescriber reference data; and
receiving, from the pharmacy switch server, a second pharmacy claim transaction message associated with a second pharmacy claim transaction for a prescription for a same prescribed product as the first pharmacy claim transaction and prescribed by the same prescriber as the first pharmacy claim transaction;
querying the reference database with the prescriber identifier to access the prescriber reference data associated with the prescriber;
detecting the prescribed approved status in the prescriber reference data; and
automatically appending an approval data element to the second pharmacy claim transaction message to generate a second PA approval message.

11. The computer-implemented method of claim 1, further comprising monitoring, for the prescriber, a progress toward at least one prescriber approved status using the prescriber reference data.

12. A prior authorization pre-approval (paPA) computing device, the paPA computing device comprising at least one processor in communication with at least one memory device storing computer-executable instructions that, when executed by the at least one processor, cause the at least one processor to:
receive, from a pharmacy switch server, a pharmacy claim transaction message associated with a pharmacy claim transaction for a prescription that has satisfied a prior authorization (PA) trigger condition, the pharmacy claim transaction message including (i) a plan identifier associated with a healthcare plan under which the prescription is submitted at an originating pharmacy, and (ii) a prescriber identifier associated with a prescriber of the prescription, wherein the pharmacy switch server is associated with a plurality of pharmacies and a plurality of pharmacy claims processors;
query a reference database with the prescriber identifier and the plan identifier to access prescriber reference data associated with the prescriber of the prescription, healthcare plan provisions of the healthcare plan, and jurisdictional regulations;
apply a set of pre-approval rules to the prescriber reference data, the healthcare plan provisions, and the jurisdictional regulations, the set of pre-approval rules defining pre-approval conditions for pharmacy PAS;
when the pre-approval conditions are met:
automatically append an approval data element to the pharmacy claim transaction message to generate a PA approval message; and
transmit the PA approval message to the pharmacy switch server for forwarding to an originating pharmacy to fulfill the prescription or to a first pharmacy claims processor for further adjudication of the pharmacy claim transaction; and
write a new record to the reference database including an indication of whether the pharmacy claim transaction for the prescription satisfied the pre-approval conditions.

13. The paPA computing device of claim 12, wherein the computer-executable instructions further cause the processor to build the reference database based on prescriber reference data provided by at least one of: each of the plurality of pharmacy claims processors, or (ii) a plurality of pharmacy switch servers including the pharmacy switch server.

14. The paPA computing device of claim 12, wherein the computer-executable instructions further cause the processor to generate the set of pre-approval rules based on jurisdictional regulations and healthcare plan provisions provided by at least one of: (i) each of the plurality of pharmacy claims processors, or (ii) a plurality of pharmacy switch servers including the pharmacy switch server.

15. The paPA computing device of claim 12, wherein the computer-executable instructions further cause the processor to:
when the pre-approval conditions are not met:
automatically append a PA initiation data element to the pharmacy claim transaction message to generate a PA initiation message; and
transmit a PA rejection message to the pharmacy switch server for forwarding to the originating pharmacy, to the first pharmacy claims processor to initiate manual PA processing of the pharmacy claim transaction, or to an electronic PA vendor to initiate manual PA processing of the pharmacy claim transaction by transmitting one or more PA forms to the prescriber.

16. The paPA computing device of claim 12, wherein the computer-executable instructions further cause the processor to assign a transaction identifier associated with the pharmacy claim transaction, wherein the new record includes the transaction identifier.

17. The paPA computing device of claim 12, wherein the computer-executable instructions further cause the processor to:
store, in the reference database, for each respective prescriber of a plurality of prescribers including the prescriber, at least one of: a number of PAs approved in a reference period of time before the pharmacy claim transaction was submitted, or a percentage of PAs approved in the reference period of time, for each respective prescribed product of a plurality of prescribed products, for each respective jurisdiction of a plurality of jurisdictions, and for respective provisions of each healthcare plan of a plurality of healthcare plans; and
store, in the reference database, a pre-approval rule of the set of pre-approval rules defining a minimum threshold number of PAs approved or a minimum percentage of PAs approved as established by at least one of the healthcare plan provisions or jurisdictional regulations.

18. The paPA computing device of claim 17, wherein the computer-executable instructions further cause the processor to:
store, in the reference database, the respective healthcare plan provisions for each of a plurality of healthcare plans, the respective jurisdictional provisions for each of a plurality of jurisdictions, a national provider identifier, a plurality of prescription drug identifiers, a quantity, a quantity per days value, and a number of refills;
generate one or more pre-approval rules of the set of pre-approval rules based on the healthcare plan provisions; and
generate one or more pre-approval rules of the set of pre-approval rules based on the jurisdictional regulations.

19. The paPA computing device of claim 12, wherein the computer-executable instructions further cause the processor to, when the pre-approval conditions are met, assign a prescriber approved status to the prescriber reference data.

20. The paPA computing device of claim 19, wherein the pharmacy claim transaction is a first pharmacy claim transaction, and wherein the computer-executable instructions further cause the processor to:
receive, from the pharmacy switch server, a second pharmacy claim transaction message associated with a second pharmacy claim transaction for a prescription for a same prescribed product as the first pharmacy claim transaction and prescribed by the same prescriber as the first pharmacy claim transaction;
query the reference database with the prescriber identifier to access the prescriber reference data associated with the prescriber;
detect the prescribed approved status in the prescriber reference data; and
automatically append an approval data element to the second pharmacy claim transaction message to generate a second PA approval message.
